(19) United States
(12) Patent Application Publication
(10) Pub. No.: US 2015/0209310 A1
(43) Pub. Date: Jul. 30, 2015

(54) NACA FOR THE TREATMENT OF CHRONIC OR LOW IMPACT BRAIN TRAUMA

(71) Applicant: Brighton Biotech, Inc., Fort Worth, TX (US)

(72) Inventor: Craig S. Rosenfeld, Dallas, TX (US)

(21) Appl. No.: 14/602,848

(22) Filed: Jan. 22, 2015

Related U.S. Application Data
(60) Provisional application No. 61/931,305, filed on Jan. 24, 2014.

(51) Int. Cl. A61K 31/16; A61K 49/00

(52) U.S. Cl. CPC A61K 31/16; A61K 49/0004

(57) ABSTRACT

The present invention includes compositions and methods for treating a human subject in need of treatment for traumatic brain injury or neurological damage resulting from exposure to one or more low-energy impacts, comprising administering to the human subject an effective dose of N-acetylcysteine amide (NACA), or a pharmaceutically acceptable salt or ester thereof, thereby treating the human subject in need of treatment for traumatic brain injury or spinal cord injury resulting from exposure to one or more chronic or low-energy impacts.

### CROSS-REFERENCE TO RELATED APPLICATIONS

This non-provisional Patent Application claims priority to U.S. Provisional Patent Application Ser. No. 61/931,305, filed Jan. 24, 2014, entitled “NACA for The Treatment of Chronic or Low Impact Brain Trauma,” the contents of which is incorporated by reference herein in its entirety.

### TECHNICAL FIELD OF THE INVENTION

The present invention relates in general to the field of treatments for traumatic brain injury, and more particularly, to the use of an effective amount of N-acetylcysteine amide (NACA) for the treatment of chronic or low impact brain trauma.

### STATEMENT OF FEDERALLY FUNDED RESEARCH

None.

### BACKGROUND OF THE INVENTION

Without limiting the scope of the invention, its background is described in connection with brain injuries.

U.S. Pat. No. 8,354,449, issued to Goldstein, entitled, “N-acetylcysteine amide (NAC amide) for the treatment of diseases and conditions associated with oxidative stress”, claims a method of treating a human subject in need of treatment for traumatic brain injury or spinal cord injury resulting from exposure to a high-energy impulse blast, comprising administering to the human subject an effective dose of N-acetylcysteine amide (NAC Amide), or a pharmaceutically acceptable salt or ester thereof, thereby treating the human subject in need of treatment for traumatic brain injury or spinal cord injury resulting from exposure to a high-energy impulse blast, which is an abrupt rise in atmospheric pressure lasting a very short time, e.g., a 140 kPa concussive air blast.

United States Patent Application No. 20090234011 filed by Goldstein, entitled “N-acetylcysteine amide (NAC amide) for the treatment of diseases and conditions associated with oxidative stress”, is said to teach methods and compositions comprising N-acetylcysteine amide (NAC amide) and derivatives thereof are used in treatments and therapies for human and non-human mammalian diseases, disorders, conditions and pathologies. Also discussed are pharmaceutically or physiologically acceptable compositions of NAC amide or derivatives thereof are administered alone, or in combination with other suitable agents, to reduce, prevent, or counteract oxidative stress and free radical oxidant formation and overproduction in cells and tissues, as well as to provide a new source of glutathione.

Methods and compositions comprising N-acetylcysteine (NAC) and derivatives thereof are used in treatments and therapies for human and non-human mammalian diseases, disorders, conditions and pathologies. Pharmaceutically or physiologically acceptable compositions of NAC or derivatives thereof are administered alone, or in combination with other suitable agents, to reduce, prevent, or counteract oxidative stress and free radical oxidant formation and overproduction in cells and tissues, as well as to provide a new source of glutathione. However, a critical challenge to the use of NAC is its inability to cross the blood-brain barrier.

### SUMMARY OF THE INVENTION

The present invention includes methods and compositions for treating a human subject in need of treatment for traumatic brain injury or neurological damage resulting from exposure to one or more low-energy impacts, comprising administering to the human subject an effective dose of N-acetylcysteine amide (NACA or NAC Amide), or a pharmaceutically acceptable salt or ester thereof, thereby treating the human subject in need of treatment for traumatic brain injury or spinal cord injury resulting from exposure to one or more low-energy impacts. In one aspect, the NACA is administered prophylactically before the one or more low-energy impacts. In another aspect, the dose for administration is 100, 150, 150, 300, 333, 400, 500, 600, 700, 750, 800, 900, 1,000, 2,500, 5,000, 7,500, or 10,000 mg per dose. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per day. In another aspect, the NACA is delivered orally via a mini-tablet, capsule, tablet, effervescent, dual release, mixed release, sachet, powder, or liquid. In another aspect, the NACA is administered orally, subcutaneously, intravenously, intramuscularly, intrasternally, or intraperitoneally. In another aspect, the NACA is administered for immediate release orally, via inhalation, topically, or intranasally. In another aspect, the NACA is administered before and/or after exposure to one or more low-energy impacts, a low-energy dose of radiation. In another aspect, the NACA is administered before and/or after exposure to a low-energy impact. In another aspect, the low-energy impacts are not high-energy concussive air blasts, high-energy sound blasts, or high-energy radiation exposure. In another aspect, the low-energy impulse impacts are the result of one or more low-energy impacts during a sport, combat, loss of balance, an accident, shaken-baby syndrome, or repetitive strikes to the brain or brainstem. In another aspect, the wherein the NACA is administered prophylactically prior to one or more low-energy impulse impacts.

Another embodiment of the present invention includes a method of treating a subject in need of treatment for traumatic brain injury or neurological damage resulting from exposure to one or more low-energy impacts, comprising: identifying a subject suspected of having received one or more low-energy impacts to the brain, thereby causing at least one adverse neurological symptom; and administering to the human subject an effective dose of N-acetylcysteine amide (NACA), or a pharmaceutically acceptable salt or ester thereof, thereby treating the human subject in need of treatment for traumatic brain injury or spinal cord injury resulting from exposure to the one or more low-energy impacts. In one aspect, the NACA is administered prophylactically before the one or more low-energy impacts. In another aspect, the dose for administration is 100, 150, 150, 300, 333, 400, 500, 600, 700, 750, 800, 900, 1,000, 2,500, 5,000, 7,500, or 10,000 mg per day. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, the wherein the NACA is delivered orally via a mini-tablet, capsule, tablet, effervescent, dual release, mixed release, sachet, powder, or liquid. In another aspect, the NACA is administered orally, subcutaneously, intravenously, intramuscularly, intrasternally, or intraperitoneally. In another aspect, the NACA is administered for immediate release orally, via inhalation, topically, or intranasally. In another aspect, the NACA is administered before and/or after exposure to one or more low-energy impacts, a low-energy dose of radiation. In another aspect, the NACA is administered before and/or after exposure to a low-energy impact. In another aspect, the low-energy impacts are not high-energy concussive air blasts, high-energy sound blasts, or high-energy radiation exposure. In another aspect, the low-energy impulse impacts are the result of one or more low-energy impacts during a sport, combat, loss of balance, an accident, shaken-baby syndrome, or repetitive strikes to the brain or brainstem.

Yet another embodiment of the present invention includes a method to evaluate a candidate drug believed to be useful in treating a traumatic brain injury, the method comprising: (a) measuring the extent of the traumatic brain injury from a subject suspected of having traumatic brain injury from a set of patients; (b) administering NACA to a first subset of the patients, and a placebo to a second subset of the patients; (c) repeating step (a) after the administration of the NACA or the placebo; and (d) determining if the candidate drug reduces the number of symptoms of traumatic brain injury that have been administered the NACA that is statistically significant as compared to any reduction occurring in the second subset of patients, wherein a statistically significant reduction indicates that the candidate drug is useful in treating the traumatic brain injury. In one aspect, the dose for administration is 100, 150, 150, 300, 333, 400, 500, 600, 700, 750, 800, 900, 1,000, 2,500, 5,000, 7,500, or 10,000 mg per day. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, the NACA is delivered orally via a mini-tablet, capsule, tablet, effervescent, dual release, mixed release, sachet, powder, or liquid. In another aspect, the NACA is administered orally, subcutaneously, intravenously, intramuscularly, intrasternally, or intraperitoneally. In another aspect, the NACA is administered for immediate release orally, via inhalation, topically, or intranasally. In another aspect, the NACA is administered before and/or after exposure to one or more low-energy impacts, a low-energy dose of radiation. In another aspect, the NACA is administered before and/or after exposure to one or more low-energy impacts. In another aspect, the low-energy impacts are not high-energy concussive air blasts, high-energy sound blasts, or high-energy radiation exposure. In another aspect, the low-energy impulse impacts are the result of one or more low-energy impacts during a sport, combat, loss of balance, an accident, shaken-baby syndrome, or repetitive strikes to the brain or brainstem.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “low-energy impact” refers to one or more low energy impacts that alone or in combination lead to traumatic brain injury. Non-limiting examples of low-energy impacts include common sports impacts (e.g., football, soccer, hockey, baseball, basketball, racketball, squash, cycling, skiing, cheerleading, skydiving, cave diving, spelunking, ziplining, bungie-jumping), impacts resulting from falls, accidents (e.g., car accidents), shaking (e.g., shaken baby syndrome), assaults, or other trauma in which the brain is compressed against the interior of the skull. Often, these impacts occur despite at least partially blocking the impact with a helmet or other head protection. Non-limiting examples of low-energy impacts include 2, 3, 4, 5, 6, 7, 8, 9, 10, 11, 12, 13, 14, 15, 16, 17, 18, 19, 20, 25, 30, 35, 40, 50 or more low-energy impacts, that alone are not sufficient to cause measurable brain trauma, but in the aggregate lead to traumatic brain injury. The term low-energy impact does not encompass, as is in contrast to, impacts such as high decibel trauma caused to the brain, or high-energy impacts such as those caused by, e.g., explosions or radiation, in which a single impact to the brain is sufficient to cause significant, permanent or semi-permanent brain damage.

As used herein, the term “traumatic brain injury”, “TBI”, “chronic traumatic encephalopathy”, or “CTE” refers to nondegenerative, noncongenital insults to the brain from a low-energy impact such as external mechanical force, possibly leading to temporary (and/or in the aggregate permanent) impairment of cognitive, physical, and psychosocial functions, and/or an associated diminished or altered state of consciousness. Non-limiting examples of how TBI or CTE can be determined or measured include, e.g., the Glasgow Coma Scale, the Head Injury Interdisciplinary Special Interest Group of the American Congress of Rehabilitation Medicine, the Simplified Motor Score (SMS), or the National Institutes of Health Traumatic Coma Data Bank. TBI can include a wide variety of low-energy or low-impact bumps, blows, or jolts to the head or a penetrating head injury that disrupts the normal function of the brain, which can include up to a concussion, unconsciousness, and/or amnesia after the injury.

As used herein, the term “treatment” or “treating” refers to administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology), including but not limited to, “traumatic brain injury”, “TBI”, “chronic traumatic encephalopathy”, or “CTE”. The term “controlling” includes preventing treating, eradicating, ameliorating or otherwise reducing the severity of the condition being controlled.

The terms “effective amount” or “therapeutically effective amount” indicates that the amount of the subject compound that will elicit the biological or medical response of a tissue, system, animal or human that is being sought by the researcher, veterinarian, medical doctor or other clinician, specifically, “traumatic brain injury”, “TBI”, “chronic traumatic encephalopathy”, or “CTE”. As used herein, the term “treatment” refers to the treatment of the mentioned conditions, particularly in a patient who demonstrates symptoms of the disease or disorder.

As used herein, the term “enveloped drug” or “enveloped pharmaceutical” refers to a NACA in a capsule, a suppository, a gel cap, a softgel, a lozenge, a sachet or even a fast dissolving wafer. As used herein the term “carrier” is used to describe a substance, whether biodegradable or not, that is physiologically acceptable for human or animal use and may be pharmacologically active or inactive.

As used herein, the term “immediate release” refers to a release profile to effect delivery of NACA as soon as possible, that is, as soon as practically made available to an animal, whether in active form, as a precursor and/or as a metabolite. Immediate release may also be defined functionally as the release of over 80 to 90 percent (%) of the active ingredient (NACA) within about 60, 90, 100 or 120 minutes or less. Immediate release as used herein may also be defined as making the NACA available to the patient or subject regardless of uptake. Immediate release formulations of NACA on a carrier, such as rolled or compressed beads, may be formulated such that the surface area is maximized on beads and the NACA is exposed immediately. The immediate release formulations may also include effervescing NACA that cause the disintegration of the structure integrity of NACA and carrier such that release of the active is maximized. Various immediate release dosage forms for oral administration may be designed readily by one of skill in art to achieve drug delivery to the stomach and small intestine, depending upon the choice of compression, adhesive materials and/or beading.

As used herein, the terms “extended release” and “delayed release” refer to a release profile to effect delivery of NACA over an extended period of time, defined herein as being between about 60 minutes and about 2, 4, 6 or even 8 hours. Extended release may also be defined functionally as the release of over 80 to 90 percent (%) of NACA after about 60 minutes and about 2, 4, 6 or even 8 hours. Extended release as used herein may also be defined as making NACA available to the patient or subject regardless of uptake. Various extended release dosage forms may be designed readily by one of skill in art as disclosed herein to achieve delivery to both the small and large intestines, to only the small intestine, or to only the large intestine, depending upon the choice of coating materials and/or coating thickness.

As used herein, the terms “extended release” and “delayed release” refers to those formulations that may be prepared and delivered so that release is accomplished at some generally predictable location in the lower intestinal tract more distal to that which would have been accomplished if there had been no delayed release alterations. A method for delay of release is, e.g., a coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers and compatible mixtures thereof may be used to provide the coating for the delayed or the extended release of NACA, and some of their properties, include, but are not limited to: shellac, also called purified lac, a refined product obtained from the resinous secretion of an insect. This coating dissolves in media of pH >7.

The present pharmaceutical composition may also be provided in a variety of dosage forms, e.g., solution, suspension, cream, ointment, lotion, capsule, caplet, softgel, gelcap, suppository, enema, elixir, syrup, emulsion, film, granule, gum, insert, jelly, foam, paste, pastille, pellet, spray, troche, lozenge, disk, magma, poultice, or wafer and the like.

For gelcap preparations, the pharmaceutical formulation may include oils, e.g.: (1) fixed oils, such as peanut oil, sesame oil, cottonseed oil, corn oil and olive oil; (2) fatty acids, such as oleic acid, stearic acid and isostearic acid; and fatty acid esters, such as ethyl oleate, isopropyl myristate, fatty acid glycerides and acetylated fatty acid glycerides; (3) alcohols, such as ethanol, isopropanol, hexadecyl alcohol, glycerol and propylene glycol; (4) glycerol ketals, such as 2,2-dimethyl-1,3-dioxolane-4-methanol; (5) ethers, such as poly(ethylene glycol) 450; (6) petroleum hydrocarbons, such as mineral oil and petrolatum; and (7) water, or with mixtures thereof; with or without the addition of a pharmaceutically suitable surfactant, suspending agent or emulsifying agent.

For oral, buccal, and sublingual administration, the pharmaceutical composition of the invention may be administered as either solutions or suspensions in the form of gelcaps, caplets, tablets, capsules or powders. For rectal administration, the compounds of the invention may be administered in the form of suppositories, ointments, enemas, tablets and creams for release of compound in the intestines, sigmoid flexure and/or rectum. For example, when making a suppository a beeswax/glycerol composition may be used to form a body meltable suppository for transrectal or transurethral delivery.

It is contemplated that the “immediate release” of NACA can also be formulated as, e.g., freeze dried, rotary dried or spray dried powders; amorphous or crystalline powders; granules, precipitates or particulates. The immediate NACA may be either free-flowing or compressed. The pharmaceutical formulation may further include, e.g., water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

Effervescent pharmaceutical formulations are well known in the art and include, generally, an acid such as citric acid or a mono or dihydrogen salt thereof and a carbon dioxide source such as a carbonate or hydrogen carbonate alkali metal salt, such as sodium hydrogen carbonate. The acid and the carbon dioxide source do not react together when dry but combine to release carbon dioxide and an effervescent effect in the presence of water. The effervescent pharmaceutical compositions for use with the present invention may be in the form of a tablet for dissolving in water or a dispersible powder for sprinkling onto water, prior to administration. The acid and the carbon dioxide source are blended together during manufacture of the composition in the absence of water to prevent premature effervescence.

Effervescent pharmaceutical compositions may be in the form of a tablet for dissolving in water or a dispersible powder for sprinkling onto water, prior to administration. The components of the couple are blended together during manufacture of the composition. Suitable pharmaceutical formulations include effervescent tablets and sachets containing water dispersible powders. Effervescent pharmaceutical formulations according to the present invention may be prepared by blending together the granulates formed by roller compaction with other components prior to processing into, e.g., beads. Roller compaction may also be extended to include other components, such as one or more active ingredients and non-active ingredients or excipients such as lubricants, disintegrants, flavors and sweeteners. For capsule, final processing may include introducing the beads into the capsules using an encapsulation machine.

For example, monosodium citrate and sodium bicarbonate, are blended together and then roller compacted, in the absence of water, to form flakes that are then crushed to give granulates. The granulates are then combined with the NACA or salt thereof, conventional beading or filling agents and, optionally, sweeteners, flavors and lubricants. The granules are then filled together under controlled ambient conditions, to form beads or capsules, respectively. Suitable mini-tablets will have a hardness in the range 6 to 12 Kp. The hardness of the final tablets is influenced by the linear roller compaction strength used in preparing the granulates, which are influenced by the particle size of the monosodium hydrogen carbonate and sodium hydrogen carbonate. For smaller particle sizes, a linear roller compaction strength of about 15 to 20 KN/cm may be used.

The pharmaceutical composition may also be administered as a liquid suspension or solution using a sterile liquid, e.g., oil, water, an alcohol, or mixtures thereof, with or without the addition of a pharmaceutically suitable surfactant, suspending agent, or emulsifying agent for oral or parenteral administration. For liquid preparations, the pharmaceutical composition can be formulated suitably with oils, for example, fixed oils, such as peanut oil, sesame oil, cottonseed oil, corn oil and olive oil; fatty acids, such as oleic acid, stearic acid and isotearic acid; and fatty acid esters, such as ethyl oleate, isopropyl myristate, fatty acid glycerides and acetylated fatty acid glycerides; with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

The immediate release NACA of the present invention may be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art. The extended release NACA may be provided in the form of a minicapsule, a capsule, a tablet, an implant, a troche, a lozenge (minitablet), a temporary or permanent suspension, a pellet, a bead, a pill, a strip or a sachet.

The pharmaceutical composition and/or the solid carrier particles can be coated with one or more enteric coatings, seal coatings, film coatings, barrier coatings, compress coatings, fast disintegrating coatings, or enzyme degradable coatings. Multiple coatings may be applied for desired performance. Further, NACA may be provided for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The compositions may be formulated for oral, nasal, buccal, ocular, urethral, transmucosal, vaginal, topical or rectal delivery, although oral delivery is used mostly.

When formulated as a capsule, the capsule can be a hard or soft gelatin capsule, a starch capsule, or a cellulosic capsule. Although not limited to capsules, such dosage forms may be further coated with, for example, a seal coating, an enteric coating, an extended release coating, or a targeted delayed release coating.

As used herein, the term “enteric coating” refers to a mixture of pharmaceutically acceptable excipients that is applied to, combined with, mixed with or otherwise added to the carrier or composition to coat the NACA. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; easy application as a coating (substrate friendly); and economical practicality.

Dosage forms of the compositions of the present invention can also be formulated as enteric coated delayed release oral dosage forms, i.e., as an oral dosage form of a pharmaceutical composition as described herein that uses an enteric coating to effect release in the lower gastrointestinal tract. The enteric coated dosage form may be a compressed or molded or extruded tablet/mold (coated or uncoated) containing granules, pellets, beads or particles of NACA and/or other composition components, which are themselves coated or uncoated. The enteric coated oral dosage form may also be a capsule (coated or uncoated) containing pellets, beads or granules of the solid carrier or the composition, which are themselves coated or uncoated.

The coating may also contain a plasticizer and possibly other coating excipients such as colorants, talc, and/or magnesium stearate, which are well known in the art. Suitable plasticizers include: triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate.

In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the lower intestinal tract is reached.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

Immediate release coating of solid carriers is commonly used to improve product elegance as well as for a moisture barrier, and taste and odor masking Rapid breakdown of the film in gastric media is important, leading to effective disintegration and dissolution. Eudragit RD 100 (Rohm) is an example of such a coating. It is a combination of a water insoluble cationic methacrylate copolymer with a water-soluble cellulose ether. In powder form, it is readily dispensible into an easily sprayable suspension that dries to leave a smooth film. Such films rapidly disintegrate in aqueous media at a rate that is independent of pH and film thickness.

N-acetyl-L-cysteine (NAC) is a well-known thiol-containing antioxidant that has been approved by FDA as an antidote for acetaminophen intoxication and has been used in the clinic for over 50 years for indications including mucolytic therapy for respiratory conditions with excessive and/or thick mucus production, prevention of radiocontrast-induced nephrotoxicity, treatment of cyclophosphamide-induced hemorrhagic cystitis, and reduction of symptoms of both schizophrenia and bipolar disease [Kelly 1998]. NAC's effectiveness has been primarily attributed to its ability to reduce extracellular cystine to cysteine and as a source of sulfhydryl groups [DeVries et al 1993]. However, use of NAC has been limited by several drawbacks, most importantly low membrane penetration and <10% systemic bioavailability for oral formulations [Ates et al 2008; Kahns & Bundgaard 1993]. Disulfide linkage to proteins and deacetylation of NAC in the intestinal mucosa and lumen are probably the greatest factors in the low oral bioavailability of NAC.

N-acetylcysteine amide (NACA), the amide form of NAC, can act as a carrier of NAC. Due to its neutral carboxyl group, NACA has increased lipophilicity and, therefore, greater cell permeability than NAC [Atlas et al 1999]. NACA has been shown to increase cellular levels of glutathione (in cells with decreased levels) either by providing the limiting substrate, cysteine, for glutathione biosynthesis or by reducing its oxidized form, glutathione disulfide, in a thiol exchange reaction [Grinberg et al 2005; Bartov et al 2006]. Experimental studies have shown that NACA results in remarkable restoration of intracellular thiols, a more effective protection against hemoglobin oxidation, and a substantial reduction of intracellular oxidation compared with NAC [Grinberg et at 2005; Bartov et at 2006].

NACA acts by scavenging existing reactive oxygen species while halting production of reactive oxygen species by reversing lipid peroxidation. Additionally, NACA functioned by increasing the levels of reduced glutathione and the phase II detoxification enzyme glutathione peroxidase. Treatment of mice exposed to phototoxic doses of light with NACA maintained retinal pigment epithelial cell integrity and prevented outer nuclear layer cell death as examined by histopathologic methods and rescued photoreceptor function as measured by electroretinography. Together, these observations indicate that NACA protects against oxidative induced-stress and demonstrates that NACA effectively and efficiently crosses the blood-brain barrier.

NACA drug product may be formulated as a co-crystal with a GRAS (generally recognized as safe) excipient in an attempt to provide an increased plasma half-life for NAC; specifications for an oral formulation of NACA drug product (in hard gelatin capsules) can also be used; NACA for treatment of chronic or low impact brain trauma may be initiated using an oral dose of between 100-3,000, 200-2,500, 300-2000, 400-1,500, 500-1,000, 600-900, 700-800 mg of NACA per day. The dosage can be increased or decreased depending on the result of the treatment and its effect on the chronic or low impact brain trauma. In one specific dose, the NACA is provided for immediate release for acute treatment of an initial low energy impact, followed by slow or extended release for the follow-on treatment of the chronic production of oxidative-stress due to the low impact brain trauma.

The present inventors have found that NACA finds particular uses for the treatment of chronic or low impact brain trauma. Experimental evidence has shown that NACA is a carrier for NAC and that only NAC is detected in plasma following administration of NACA, using a sensitive analytical method that is capable of differentiating NAC and NACA [Wu et at 2006].

The present inventors have established analytical methods and acceptance criteria for NACA drug substance for description (appearance), identification (¹H-NMR spectrum and HPLC retention time), assay (content by HPLC), and impurities (HPLC for related substances; headspace GC for residual solvents; and titrations for water content and heavy metals).

The present invention includes a NACA “pharmaceutical co-crystal”, as defined in the FDA Guidance for Industry Regulatory Classification of Pharmaceutical Co-Crystals, using an excipient that is commonly used in human applications. The NACA “pharmaceutical co-crystal” includes the NACA and excipient compounds in the co-crystal exist in their neutral states and interact via nonionic (versus ionic) interactions, based on either relative pKa values or various orthogonal approaches using spectroscopic tools. The NACA will generally completely dissociate from its excipient prior to reaching the site of action for pharmacologic activity.

In one comparative example, NAC is used for treatment of acetaminophen overdose using 140 mg/kg as the loading dose, then 70 mg/kg every 4 hours for 17 doses, starting 4 hours after the loading dose. In clinical studies, NAC has been administered orally from 400 to 1000 mg once daily and from 200 to 600 milligrams three times daily. By contrast, the present inventors have found that a NACA dose of 200, 300, 350, 400, 450, 500, 600, 650, 700, 750, 850, 900, 950, 1,000, 1,100, 1,200, 1,300, 1,400, 1,500, 1,600, 1,700, 1,800, 1,900, 2,000, 2,500, 3,000, 4,000, 5,000, or 10,000 milligrams and higher is well tolerated as a total daily dose for treatment of chronic or low impact brain trauma. Surprisingly, the present inventors have found that NACA has less toxicity in animal model systems than NAC, thus permitting the use of much higher doses for treatment of oxidative stress. As such, the present invention includes doses of NACA that exceed those of NAC, without undue effects and having a higher effectiveness, while at the same time delivering a beneficial outcome for the patient or subject provided NACA.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Ates B, Abraham L, Ercal N. Antioxidant and free radical scavenging properties of N-acetylcysteine amide (NACA) and comparison with N-acetylcysteine (NAC). Free Radic Res 2008; 42:372-377

Atlas D, Mclamed E, Offen D. Brain targeted low molecular weight hydrophobic antioxidant compounds. U.S. Pat. No. 5,874,468 (1999)

Bartov O, Sultana R, Butterfield A, Atlas D. Low molecular weight thiolamides attenuate MAPK activity and protect primary neurons from Abeta(1-42) toxicity. Brain Res 2006; 1069:198-206

Borgstrom L, Kagedal B, Paulsen O. Pharmacokinetics of N-acetylcysteine in man. Eur J Clin Pharmacol 1986; 31:217-222

DeCaro L, Ghizzi A, Costa R, et al. Pharmacokinetics and bioavailability of oral acetylcysteine in healthy volunteers. Arzneimittelforschung 1989; 39:382-386

DeVries N, DeFlora S. N-Acetyl-L-cysteine. J Cell Biochem 1993; Suppl 17F:270

Grinberg L, Fibach E, Amer J, Atlas D. N-acetylcysteine amide, a novel cell-permeating thiol, restores cellular glutathione and protects human red blood cells from oxidative stress. Free Radic Biol Med 2005; 38:136-145

Kahns A H, Bundgaard H. Prodrugs as drug delivery systems. 107. Synthesis and chemical and enzymatic hydrolysis kinetics of various mono- and diester prodrug of N-acetylcysteine. Int J Pharm 1990; 62:193-205

Keller J N, Hanni K B, Gabita S P, et al. Oxidized lipoproteins increase reactive oxygen species formation in microglia and astrocyte cell lines. Brain Res 1999; 830:10-15

Kelly G S. Clinical application of N-acetylcysteine. Alternative Medicine Review 1998; 3:114-127

Olsson B, Johansson M, Gabrielsson J, Bolme P. Pharmacokinetics and bioavailability of reduced and oxidized N-acetylcysteine. Eur J Clin Pharmacol 1988; 34:77-82

Roy A, Jana A, Yatish K, et al. Reactive oxygen species up-regulate CD11b in microglia via nitric oxide: implications for neurodegenerative diseases. Free Radic Biol Med 2008; 45:686-699

Tuson M, Garanto A, Gonzalez-Duarte R, Marfany G. Over-expression of CERKL, a gene responsible for retinitis pigmentosa in humans, protects cells from apoptosis induced by oxidative stress. Mol Vis [serial online] 2009; 15:168-180

Usui S, Komeima K, Lee S Y, et al. Increased expression of catalase and superoxide dismutase 2 reduces cone cell death in retinitis pigmentosa. Mol Ther 2009a; 17:778-786

Usui S, Overson B C, Lee S Y, et al. NADPH oxidase plays a central role in cone cell death in retinitis pigmentosa. J Neurochem 2009b; 110:1028-1037

Wu W, Goldstein G, Adams C, Matthews R H, Ercal N. Separation and quantification of N-acetyl-L-cysteine and N-acetyl-cysteine-amide by HPLC with fluorescence detection. Biomed Chromatog 2006; 20:415-422

Yoshida N, Ikeda Y, Notomi S, et al. Laboratory evidence of sustained chronic inflammatory reaction in retinitis pigmentosa. Ophthalmology 2013; 120:e5-e12

Yu D Y, Cringle S J, Su E N, Yu P K. Intraretinal oxygen levels before and after photoreceptor loss in the RCS rat. Ophthalmol Vis Sci 2000; 41:3999-4006

What is claimed is:

1. A method of treating a human subject in need of treatment for traumatic brain injury or neurological damage resulting from exposure to one or more low-energy impacts, comprising administering to the human subject an effective dose of N-acetylcysteine amide (NACA), or a pharmaceutically acceptable salt or ester thereof, thereby treating the human subject in need of treatment for traumatic brain injury or spinal cord injury resulting from exposure to one or more low-energy impacts.

2. The method of claim 1, wherein the NACA is administered prophylactically before the one or more low-energy impacts.

3. The method of claim 1, wherein the dose for administration is 100, 150, 150, 300, 333, 400, 500, 600, 700, 750, 800, 900, 1,000, 2,500, 5,000, 7,500, or 10,000 mg per day.

4. The method of claim 1, wherein the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose.

5. The method of claim 1, wherein the NACA is delivered orally via a mini-tablet, capsule, tablet, effervescent, dual release, mixed release, sachet, powder, or liquid.

6. The method of claim 1, wherein the NACA is administered orally, subcutaneously, intravenously, intramuscularly, intrasternally, or intraperitoneally.

7. The method of claim 1, wherein the NACA is administered for immediate release orally, via inhalation, topically, or intranasally.

8. The method of claim 1, wherein the NACA is administered before and/or after exposure to one or more low-energy impacts, a low-energy dose of radiation.

9. The method of claim 1, wherein the NACA is administered before and/or after exposure to a low-energy impact.

10. The method of claim 1, wherein the low-energy impacts are not high-energy concussive air blasts, high-energy sound blasts, or high-energy radiation exposure.

11. The method of claim 1, wherein the low-energy impulse impacts are the result of one or more low-energy impacts during a sport, combat, loss of balance, an accident, shaken-baby syndrome, or repetitive strikes to the brain or brainstem.

12. The method of claim 1, wherein the NACA is administered prophylactically prior to one or more low-energy impulse impacts.

13. A method of treating a subject in need of treatment for traumatic brain injury or neurological damage resulting from exposure to one or more low-energy impacts, comprising:
identifying a subject suspected of having received one or more low-energy impacts to the brain, thereby causing at least one adverse neurological symptom; and
administering to the human subject an effective dose of N-acetylcysteine amide (NACA), or a pharmaceutically acceptable salt or ester thereof, thereby treating the human subject in need of treatment for traumatic brain injury or spinal cord injury resulting from exposure to the one or more low-energy impacts.

14. The method of claim 13, wherein the NACA is administered prophylactically before the one or more low-energy impacts.

15. The method of claim 13, wherein the dose for administration is 100, 150, 150, 300, 333, 400, 500, 600, 700, 750, 800, 900, 1,000, 2,500, 5,000, 7,500, or 10,000 mg per day.

16. The method of claim 13, wherein the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose.

17. The method of claim 13, wherein the NACA is delivered orally via a mini-tablet, capsule, tablet, effervescent, dual release, mixed release, sachet, powder, or liquid.

18. The method of claim 13, wherein the NACA is administered orally, subcutaneously, intravenously, intramuscularly, intrasternally, or intraperitoneally.

19. The method of claim 13, wherein the NACA is administered for immediate release orally, via inhalation, topically, or intranasally.

20. The method of claim 13, wherein the NACA is administered before and/or after exposure to one or more low-energy impacts, a low-energy dose of radiation.

21. The method of claim 13, wherein the NACA is administered before and/or after exposure to a low-energy impact.

22. The method of claim 13, wherein the low-energy impacts are not high-energy concussive air blasts, high-energy sound blasts, or high-energy radiation exposure.

23. The method of claim 13, wherein the low-energy impulse impacts are the result of one or more low-energy impacts during a sport, combat, loss of balance, an accident, shaken-baby syndrome, or repetitive strikes to the brain or brainstem.

24. The method of claim 13, wherein the NACA is administered prophylactically prior to one or more low-energy impulse impacts.

25. A method to evaluate a candidate drug believed to be useful in treating a traumatic brain injury, the method comprising:
a) measuring the extent of the traumatic brain injury from a subject suspected of having low-energy impact traumatic brain injury from a set of patients;
b) administering NACA to a first subset of the patients, and a placebo to a second subset of the patients;
c) repeating step (a) after the administration of the NACA or the placebo; and
d) determining if the candidate drug reduces the number of symptoms of traumatic brain injury that have been administered the NACA that is statistically significant as compared to any reduction occurring in the second subset of patients, wherein a statistically significant reduction indicates that the candidate drug is useful in treating the traumatic brain injury.

26. The method of claim 25, wherein the dose for administration is 100, 150, 150, 300, 333, 400, 500, 600, 700, 750, 800, 900, 1,000, 2,500, 5,000, 7,500, or 10,000 mg per dose.

27. The method of claim 25, wherein the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose.

28. The method of claim 25, wherein the NACA is delivered orally via a mini-tablet, capsule, tablet, effervescent, dual release, mixed release, sachet, powder, or liquid.

29. The method of claim 25, wherein the NACA is administered orally, subcutaneously, intravenously, intramuscularly, intrasternally, or intraperitoneally.

30. The method of claim 25, wherein the NACA is administered for immediate release orally, via inhalation, topically, or intranasally.

31. The method of claim 25, wherein the NACA is administered before and/or after exposure to one or more low-energy impacts, a low-energy dose of radiation.

32. The method of claim 25, wherein the NACA is administered before and/or after exposure to a low-energy impacts.

33. The method of claim 25, wherein the low-energy impacts are not high-energy concussive air blasts, high-energy sound blasts, or high-energy radiation exposure.

34. The method of claim 25, wherein the low-energy impulse impacts are the result of one or more low-energy impacts during a sport, combat, loss of balance, an accident, shaken-baby syndrome, or repetitive strikes to the brain or brainstem.

35. The method of claim 25, wherein the NACA is administered prophylactically prior to one or more low-energy impulse impacts.
